(19) United States
(12) Patent Application Publication
(10) Pub. No.: US 2024/0311743 A1
(43) Pub. Date: Sep. 19, 2024

(54) SYSTEMS AND METHODS FOR IDENTIFYING EVENTS IN SUPPLY CHAIN PROCESSES

(71) Applicant: Regeneron Pharmaceuticals, Inc., Tarrytown, NY (US)

(72) Inventors: Diego De La Torre Campos, Albany, NY (US); Fletcher Dix, Clifton Park, NY (US); Jason Castle, Esperance, NY (US); Andrew Croft, Hagaman, NY (US)

(21) Appl. No.: 18/603,862

(22) Filed: Mar. 13, 2024

Related U.S. Application Data
(60) Provisional application No. 63/490,380, filed on Mar. 15, 2023.

(51) Int. Cl. G06Q 10/0832

(52) U.S. Cl. CPC G06Q 10/0832

(57) ABSTRACT

A method for identifying an event during a supply chain process that includes preparing a product during the supply chain process, determining an occurrence of the event during preparation of the product during the supply chain process, and identifying a modification to the supply chain process to inhibit occurrences of the event during subsequent preparations of the product in the supply chain process. The event causing a change of the product.

### CROSS-REFERENCE TO RELATED APPLICATIONS

This application claims priority to U.S. Application No. 63/490,380, filed Mar. 15, 2023, which is hereby incorporated by reference in its entirety.

### FIELD OF DISCLOSURE

Aspects of the present disclosure relate to systems and methods for identifying an occurrence of an event during a supply chain process, such as for a product. More specifically, embodiments of the present disclosure relate to systems and methods for detecting the occurrence of an event and determining one or more measurable parameters (e.g., a force, a pressure, a temperature, a light intensity, an impedance, etc.) experienced by a product at a particular stage of a supply chain process. The product may include an apparatus or a device, such as a medical device. The supply chain process may include one or more stages, such as manufacturing, assembly, testing, sterilizing, packaging, and/or delivery of the product.

The systems and methods of the present disclosure may identify a particular stage during the supply chain process, such as one or more periods and/or points of time, in which the event occurs. The event may be indicative of an error or fault in the supply chain process at which the product may become structurally compromised or rendered inoperable for use. The source of the event may be attributed to various characteristics or sources in the supply chain process, such as instrumentation and/or tools used for preparing and/or managing the product during the particular stage at which the event occurs. By identifying the stage of the supply chain process in which the event occurs, the systems and methods of the present disclosure may identify a modification of one or more of the characteristics or sources associated with the particular stage of the supply chain process to mitigate future occurrences of the event.

### INTRODUCTION

A preparation and/or management of a product during a supply chain process may involve manufacturing, assembling, testing, sterilizing, packaging, and/or delivering the product (e.g., a medical device, such as a syringe storing a fluid substance) in various arrangements, positions, configurations, and/or locations. In some instances, a parameter, such as a load (e.g., a force), may be inadvertently applied to the product by one or more sources associated with the supply chain process, such as manufacturing instrumentation. Application of the parameter may cause unintended modifications and/or damage to the product. In instances where the product is a medical device, and more particularly a syringe, such modifications and/or damage may result in premature failures and/or inaccurate use of the syringe by a user. In other instances, human error in handling, controlling, positioning, and/or delivering the product (e.g., a syringe) during a stage of the supply chain process may similarly entail the application of a parameter that may influence a structural integrity of the product.

The occurrence (i.e., the event) of a parameter (e.g., a load) being applied to a syringe prior to its use in administering a dose to a user may cause health and safety complications for the user (e.g., a patient). For example, occurrences of a load to the syringe may result in a misalignment of a needle of the syringe, an inadvertent release of at least a portion of a fluid substance stored in the syringe, and more, thereby causing ineffective syringes for potential use by users.

### SUMMARY

Disclosed herein are systems and methods for identifying an event during a supply chain process of a product, and particularly medical devices such as syringes storing a fluid substance, in which the event causes damage to the product. In one embodiment of the present disclosure, a method for identifying an event during a supply chain process includes preparing a product during the supply chain process; determining an occurrence of the event during preparation of the product during the supply chain process, wherein the event causes a change of the product and/or at least partially impairs the product; and identifying a modification to the supply chain process to inhibit occurrences of the event during subsequent preparations of the product.

### BRIEF DESCRIPTION OF THE DRAWINGS

The accompanying drawings, which are incorporated into and constitute a part of this specification, illustrate various exemplary embodiments and, together with the description, serve to explain principles of the disclosed embodiments. The drawings show different aspects of the present disclosure and, where appropriate, reference numerals illustrating like structures, components, materials, and/or elements in different figures are labeled similarly. It is understood that various combinations of the structures, components, and/or elements in various embodiments, other than those specifically shown, are contemplated and are within the scope of the present disclosure.

FIG. 1 depicts an exploded elevation view of an exemplary product including a medical device, according to aspects of the present disclosure.

FIG. 2 depicts a schematic of an exemplary supply chain process of the product of FIG. 1, according to aspects of the present disclosure.

FIG. 3 depicts a flowchart of an exemplary method for identifying an occurrence of an event to the product of FIG. 1 during the supply chain process of FIG. 2, according to aspects of the present disclosure.

FIG. 4 depicts a schematic of an exemplary manufacturing stage of the supply chain process of FIG. 2, according to aspects of the present disclosure.

There are many embodiments described and illustrated herein. The present disclosure is neither limited to any single aspect nor embodiment thereof, nor to any combinations and/or permutations of such aspects and/or embodiments. Each of the aspects of the present disclosure, and/or embodiments thereof, may be employed alone or in combination with one or more of the other aspects of the present disclosure and/or embodiments thereof. For the sake of brevity, many of those combinations and permutations are not discussed separately herein.

### DETAILED DESCRIPTION

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Notably, an embodiment or implementation described herein as an “example” or “exemplary” is not to be construed as preferred or advantageous, for example, over other embodiments or implementations; rather, it is intended reflect or indicate the embodiment(s) is/are one “example,” rather than “ideal.” In addition, the terms “first,” “second,” and the like, herein do not denote any order, quantity, or importance, but rather are used to distinguish an element, a structure, a step or a process from another. Moreover, the terms “a” and “an” herein do not denote a limitation of quantity, but rather denote the presence of one or more of the referenced items. Additionally, the terms “about,” “approximately,” “substantially,” and the like, when used in describing a numerical value, denote a variation of +/−10% of that value, unless specified otherwise.

Embodiments of the present disclosure may be used for any type of product, including fluid-containing products, such as liquid drug substances, liquid placebos, or other liquids (or liquid solutions) that may be dispensed in a dose form. As used herein, the term “substance” may refer to a formulated substance including an active ingredient or ingredients. In some embodiments, systems and aspects of the present disclosure can be used for apparatuses and/or devices manufactured for use with any therapies using the immune system to combat diseases, such as immunotherapies, including vaccines, allergy treatments, cancer treatments, and more. In particular, systems and aspects of the present disclosure can be used for apparatuses and/or devices manufactured for the treatment of patients with cancer (e.g., immune-oncology). However, it is also contemplated that embodiments of the present disclosure may be applicable to apparatuses and/or devices used for any therapy, liquid products, and/or any other context for which one or more substances may be stored. While medical indications are described herein, it is contemplated that non-medical apparatuses and/or devices may be used in conjunction with the systems and methods described herein. It should be understood that embodiments of the present disclosure may be used with apparatuses and/or devices that do not contain any liquid substance.

As used herein, the terms “distal” and “distally” refer to a location (or portion of a device) relatively closer to, or in the direction of, a drug delivery end of the device, and the terms “proximal” and “proximally” refer to a location (or portion of a device) relatively closer to, or in the direction of, an end of the device opposite the distal end. As used herein, the term “component,” when used in reference to a part of a product, may refer to a feature of the product suitable for serving any purpose of the product. In examples in which the product may include a medical device, the component may include a body, e.g., a barrel (such as a syringe barrel), tube, cylinder, or other portion of a device capable of storing a fluid substance. In some embodiments, a body may also include a distal end portion having a nozzle, needle, needle attachment site, and/or distal cap.

Described herein is an exemplary embodiment of a product, such as an apparatus, and in particular a medical device such as an auto-injector including a syringe. In some instances, embodiments or aspects of embodiments disclosed herein may be used with medical devices including other types of fluid-containing products, such as, but not limited to, vials, cartridges, and/or other suitable devices. In further instances, embodiments or aspects of embodiments disclosed herein may be used with various other suitable products (e.g. apparatuses and/or devices). Such systems and methods may aid to identify sources of human or automated equipment error in a supply chain process, and/or increase accuracies and efficiencies in a supply chain process, by identifying an occurrence of an event during the supply chain process, such as during one or more stages of said process (e.g., a manufacturing, an assembly, a testing, a sterilizing, a packaging, a labeling/marking, a storage, a shipment, a sale, and/or a delivery process of the product).

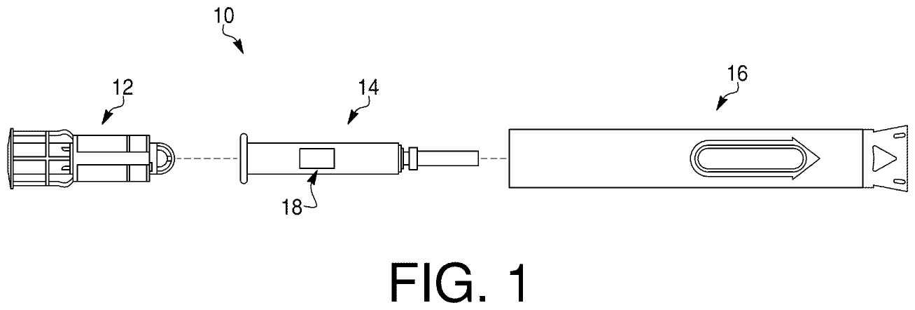

Referring now to FIG. 1, an exemplary product 10 is depicted. It should be appreciated that product 10 may include various manufactured goods, including but not limited to, a device, a tool, a machine, an instrument, a mechanism, an appliance, a gadget, an apparatus, or other various equipment. By way of example only, product 10 may include a medical device (e.g., an auto-injector) including a plurality of components, such as a first component 12, a second component 14, and a third component 16. First component 12 may include a cap that is configured to selectively couple with third component 16 to enclose one or more contents therein. Second component 14 may include a container configured to store a fluid substance, such as a medicament (e.g. a drug product). In some embodiments, the container of second component 14 may include, but is not limited to, a syringe, a barrel, a vial, a cartridge, a tube, and more.

Third component 16 may include a housing that is sized, shaped, and/or otherwise configured to at least partially receive one or more of first component 12 and second component 14 therein. In other words, third component 16 may define an outer body of product 10 for storing the internal components of product 10, such as first component 12 and second component 14. Each of first component 12, second component 14, and third component 16 may be formed of various suitable materials, including but not limited to, glass, plastic, etc. It should be understood that product 10 (e.g., an auto-injector) may include additional and/or fewer components than that shown and described herein, including but not limited to, a needle, a plunger, a biasing mechanism, and more. Alternatively, first component 12, second component 14, and/or third component 16 of product 10 may include various other shapes, sizes, arrangements, or configurations than those shown and described herein without departing from a scope of this disclosure. In some embodiments, one or more of first component 12, second component 14, and third component 16 may be omitted from product 10 entirely.

Still referring to FIG. 1, sensing device 18 may be coupled to product 10, and particularly to one or more of first component 12, second component 14, and/or third component 16. It should be understood that sensing device 18 may be selectively coupled to product 10, such that sensing device 18 is not a component of product 10. Stated differently, sensing device 18 may be removably attached to product 10 for purposes of identifying the occurrence of an event during a supply chain process of product 10 according to aspects of the present disclosure, such that sensing device 18 is not an ordinary feature of product 10. Moreover, though only a single sensing device 18 is depicted, those of ordinary skill will understand that any suitable number of sensing devices 18 may be provided within the scope of this disclosure. For example, two, three, four, or any suitable number of sensing devices may be provided, according to the scope of this disclosure.

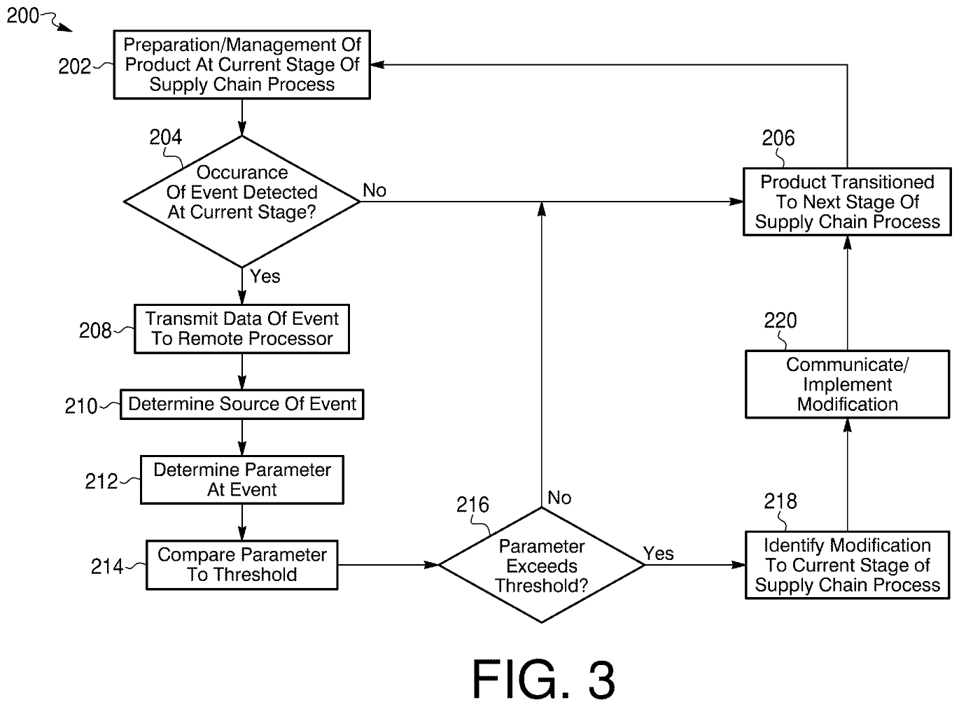

Sensing device 18 may be coupled to and/or inserted within second component 14 in the present example. In other examples, sensing device 18 may be positioned on and/or within first component 12 and/or third component 16 in lieu of second component 14. It should be appreciated that including sensing device 18 in product 10 may render product 10 as a “test” or “experimental” version of product 10 for administering the methods described in further detail herein (see FIG. 3). In other embodiments, sensing device 18 may be an ordinary feature of product 10.

Sensing device 18 may be configured to detect and/or measure one or more parameters indicative of a characteristic and/or measurable parameter experienced by product 10. For example, sensing device 18 may include a force sensor that is configured to detect and/or measure a force load applied to product 10. In another example, sensing device 18 may include an accelerometer configured to detect and/or measure an acceleration, agitation, spinning, and/or vibration of product 10. By way of further example, sensing device 18 may include a gyroscope configured to detect and measure an angular velocity of product 10. In other examples, sensing device 18 may include a temperature probe, a pressure (e.g., piezoelectric) sensor, a fluid sensor, a vaporized hydrogen peroxide (VHP) sensor, a photometer (light) sensor, a photodetector, a chemical (biological) sensor/indicator, and more. It should be understood that sensing device 18 may be configured to detect and/or measure various other suitable parameters (e.g. a rotation, a tilt, a compression, etc.) than those described herein without departing from a scope of this disclosure.

Still referring to FIG. 1, sensing device 18 may include a plurality of sensors disposed on one or more components of product 10, with each of the plurality of sensors being configured to detect and measure the same or different parameters. For example, sensing device 18 may include a sensor array ranging from approximately 16 to 32 sensors that may be coupled to a single or multiple components of product 10. In other embodiments, a single sensing device 18 may be configured and operable to detect and measure two or more parameters.

Although product 10 is shown and described herein as a medical device, and particularly an auto-injector, that has a particular configuration and arrangement of assembled components, it should be understood that product 10 may include various other medical devices, medical packages, or non-medical apparatuses such that the systems and methods described herein are not limited to auto-injectors. By way of illustrative example only, other exemplary medical products may include, e.g., suppository applicators and medication, transdermal drug delivery devices, medical implants, needles, cannulas, medical instruments, blister packs, boxes, and more.

Still referring to FIG. 1, and as described in detail herein, sensing device 18 may be operable to measure a parameter(s) experienced by product 10 from one or more sources (e.g., objects, surfaces, etc.) located in a surrounding environment of product 10 during a supply chain process of product 10. The parameter may cause a corresponding defect or damage to product 10, and may originate from one or more sources that interact with product 10 during its preparation and/or management in the supply chain process. In some embodiments, sensing device 18 may be operable to communicate with one or more remote systems or computers via various suitable communication protocols. For example, sensing device 18 may be operable to communicate with a remote system or computer (not shown) via a network that may be implemented as a wireless network (e.g. Wi-Fi), a wired network (e.g., Ethernet), a local area network (LAN), a Wide Area Network (WANs), Bluetooth, Near Field Communication (NFC), cellular satellite, or any other type of network(s) capable of providing communication between sensing device 18 and a remote system.

The remote system or computer may be configured and operable to carry out one or more steps of the methods shown and described herein according to the present disclosure, such as method 200 of FIG. 3. Although not shown, it should be understood that the remote system may include one or more computer hardware platforms having a central processing unit (“CPU”) in the form of one or more processors for executing computer-readable instructions according to the exemplary embodiments of the present disclosure. The one or more computer hardware platforms may also include a data storage for various data files to be processed and/or communicated, such as data from sensing device 18.

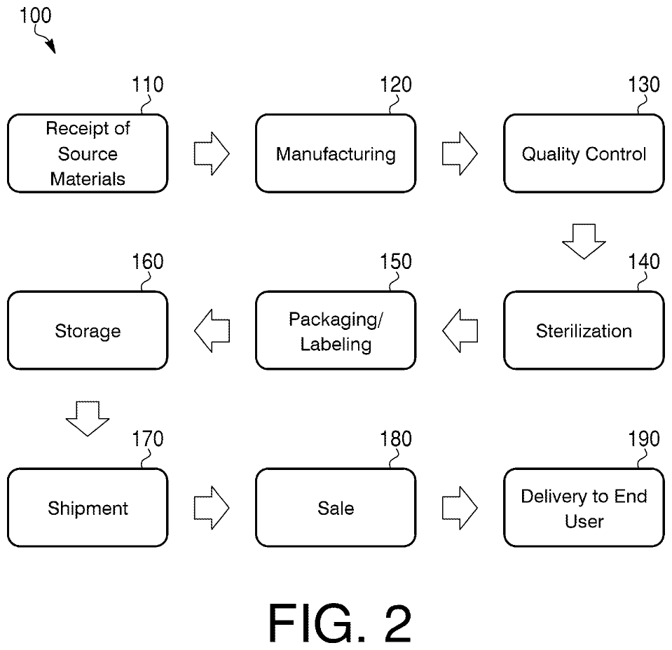

Referring now to FIG. 2, an exemplary supply chain process 100 of product 10 is depicted. Supply chain process 100 (hereinafter “process 100”) may include one or more stages or sub-processes as shown and described herein. It should be appreciated that the stages of process 100 are merely illustrative, such that additional and/or fewer stages may be included in process 100 without departing from a scope of this disclosure. Additionally, some stages of process 100 may be omitted, combined, and/or performed in a different order than that shown and described herein while remaining consistent with this disclosure.

In the example, process 100 may include a plurality of stages, such as a first stage 110 involving receipt of source materials for producing product 10; a second stage 120 involving a manufacture and/or assembly of product 10; a third stage 130 involving a quality control testing (or other forms of testing) of product 10; a fourth stage 140 involving a sterilization or cleaning of product 10; a fifth stage 150 involving a packaging and/or labeling of product 10; a sixth stage 160 involving a storage of product 10; a seventh stage 170 involving a shipment of product 10; an eight stage 180 involving a sale of product 10; and a ninth stage involving a delivery of product 10, such as to an end user.

By way of example, the first stage 110 may include a process of receiving source materials from a supplier for producing one or more of first component 12, second component 14, third component 16 of product 10. The second stage 120 may include a process of manufacturing first component 12, second component 14, third component 16, and/or sensing device 18, and/or assembling product 10. In embodiments in which product 10 includes a syringe storing a fluid substance, the second stage 120 may involve a process of filling the fluid substance within the syringe. The third stage 130 may include a process of testing the assembled product 10, such as from a quality control standpoint, to evaluate a proper assembly and operation of product 10. It should be appreciated that the third stage 130 may involve testing product 10 to an extent suitable for, and/or in accordance with an applicable standard and/or protocol associated with, the type of product.

The fourth stage 140 may include a process of sterilizing product 10 via a sterilization method. In some embodiments, the fourth stage 140 may involve a terminal sterilization of product 10 by a sterilization method using chemicals (e.g., vaporized hydrogen peroxide (VHP), ethylene oxide, nitrogen dioxide, etc.) to remove contaminants and other biological agents present on product 10. For example, the fourth stage 140 may include positioning product 10 within a sterilization chamber configured to run sterilization cycles at predefined temperatures and pressures, and supplying sterilizing chemicals into the sterilization chamber during the cycles at adjustable concentrations. It should be understood that the term “sterilization” refers to achieving a level of sterility appropriate for product 10. In the example of product 10 including an auto-injector storing a formulated drug substance, the fourth stage 140 may include the applicable sterilization techniques for commercial distribution and use of a medical device. It should be appreciated that the fourth stage 140 may involve cleaning product 10 to an extent suitable for, and/or in accordance with an applicable standard and/or protocol associated with, the type of product.

Still referring to FIG. 2, the fifth stage 150 may include a process of packaging product 10. For example, product 10 may be packaged within a protective container that is configured to house product 10, such as a blister packaging. Additionally and/or alternatively, the fifth stage 150 may include a process of labeling/marking product 10 or a packaging of product 10. The sixth stage 160 may include a process of storing product 10, such as upon completion of the aforementioned manufacturing, testing, sterilization, and packaging processes of product 10. Product 10 may be stored in isolation or with a plurality of other products, such as in containers suitable for transporting products to a retailer for sale. At the seventh stage 170, product 10 may be shipped by a distributor to one or more locations for sale. Product 10 may be shipped via various suitable transportation means (e.g., air, ground, sea, etc.) and upon reaching its destination, product 10 may be sold to an end user (e.g., a consumer) at the eight stage 180 and subsequently delivered to the end user at the ninth stage 190.

It should be understood that product 10 may interact with various and/or numerous objects, surfaces, or personnel during each of the plurality of stages of process 100. It should be understood that the term “interact” may include any physical, chemical, or other contact or exposure (direct or indirect) with product 10. For example, one or more of the plurality of components of product 10 (see FIG. 1) may be physically handled, controlled, positioned, and/or manipulated to different configurations and/or arrangements relative to one another, or a surrounding environment, during one or more of the plurality of stages of process 100. For further example, the components of product 10 may be exposed to various elements without experiencing physical contact, such as a light (or lack thereof), a heat (or lack thereof), a fluid (e.g., air, water, gas), and more during the plurality of stages of process 100.

One or more of the plurality of stages of process 100 may involve product 10 (and particularly one or more of first component 12, second component 14, or third component 16) interacting with an object responsible for performing operations associated with the manufacture, quality control testing, sterilization, packaging, storage, shipment, sale, and/or delivery of product 10. The object may include, but is not limited to, one or more surfaces, tools, assemblies, machinery, equipment, instrumentation, systems, etc. In some embodiments, the object may be remotely controlled through an automated operation, such as by a computer. In other embodiments, the object may be manually controlled and operated by personnel in process 100.

For example, product 10 may interact with one or more personnel (e.g. humans) during one or more of the plurality of stages of process 100, such as the personnel responsible for performing operations associated with the manufacture, quality control testing, sterilization, packaging, storage, shipment, sale, or delivery of product 10. It should be appreciated that various other elements, factors, or influences may be present in process 100 and involved in one or more of the manufacture, quality control testing, sterilization, packaging, storage, shipment, sale, and delivery of product 10. That is, product 10 may have additional and/or alternative interactions with its surrounding environment during process 100 than those described herein without departing from a scope of this disclosure.

In either instance, an interaction between product 10 and an object, personnel, or other factor may cause the occurrence of an event during which product 10 may experience or encounter a parameter (e.g., a force, a pressure, a temperature, a light intensity, an impedance, etc.) at a particular stage of process 100. Stated differently, the interaction of product 10 with its surrounding environment during one or more of the plurality of stages of process 100 may involve an event in which a change in (and/or creation of) a parameter occurs to at least one of the components of product 10, the parameter being sufficient to cause possible damage (e.g. structural, mechanical, chemical, etc.) to product 10. As described herein, sensing device 18 may be configured to detect and/or measure the change and/or creation of the parameter to product 10 upon the occurrence of the event at the particular stage of process 100.

FIG. 3 depicts a flow diagram of an exemplary method 200 for identifying an occurrence of an event to a product during a supply chain process according to the present disclosure. It should be appreciated that the steps of method 200 are described herein in the context of computer-executable instructions that may be executed or implemented by a remote computing system, such as one or more remote systems or computers that are in communication with sensing device 18.

At step 202, a preparation and/or management of a product, such as product 10, may be performed at a current stage of a supply chain process, such as process 100. Stated differently, product 10 may be prepared and/or managed pursuant to the operations associated with any one of the plurality of stages of process 100 shown and described above (see FIG. 2) at step 202.

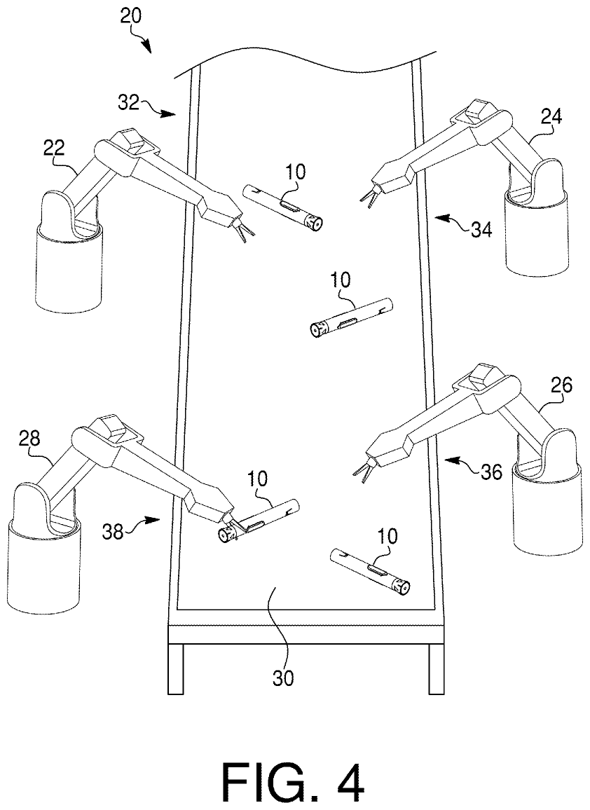

By way of example only, the current stage of process 100 at step 202 may include a manufacturing process of product 10 at the second stage 120 described above. For illustrative purposes, as seen in FIG. 4, a manufacturing environment 20 of the second stage 120 is depicted in which environment 20 may include a plurality of equipment positioned along various locations on an assembly line 30. For example, environment 20 may include a first manufacturing instrument 22 positioned adjacent to a first location 32 of the assembly line 30, a second manufacturing instrument 24 positioned adjacent to a second location 34 of the assembly line 30, a third manufacturing instrument 26 positioned adjacent to a third location 36 of the assembly line 30, and a fourth manufacturing instrument 28 positioned adjacent to a fourth location 38 of the assembly line 30. Although a particular number and arrangement of equipment is shown and described herein for the second stage 120, it should be understood that the manufacturing environment 20 is merely illustrative such that additional and/or fewer instruments (or other types of equipment) may be included in the second stage 120 of process 100, and in other suitable configurations, without departing from a scope of this disclosure.

In the example, each of the instruments in manufacturing environment 20 may be configured and operable to perform one or more operations at the respective location of the assembly line 30 for manufacturing product 10. During the second stage 120, product 10 (and particularly one or more of first component 12, second component 14, and third component 16) may interact with various objects (e.g., first manufacturing instrument 22, second manufacturing instrument 24, third manufacturing instrument 26, and fourth manufacturing instrument 28) and/or surfaces (e.g., a portion of assembly line 30 along first location 32, second location 34, third location 36, and fourth location 38). Although not shown, it should be appreciated that manufacturing environment 20 may include various other elements or factors that may interact with product 10 during the manufacturing process of the second stage 120 (e.g., a light, a temperature, a fluid, etc.).

Referring back to FIG. 3, at step 204, whether the occurrence of an event is detected at the current stage of process 100, such as by sensing device 18, is determined. As described in detail above, an event may be defined by an interaction (e.g., physical or chemical) between product 10 and the surrounding environment at the current stage of process 100, such as with an object, a personnel, or other factor. Occurrence of the event may cause sensing device 18 to detect a change in (and/or creation of) a parameter applied to product 10. That is, the occurrence of the event may cause sensing device 18 to detect a change in a property of product 10. The parameter may be to an extent or degree sufficient to cause a change in the product and/or at least partially impair product 10, such as by causing a defect or (structural) damage to one or more components of product 10.

In response to determining an event did not occur during the current stage of process 100 at step 204, method 200 may continue to step 206 in which product 10 continues to a next stage of process 100. In this instance, given that the current stage of process 100 was performed and completed without the occurrence of an event, method 200 may return to step 202 during which continued preparation and/or management of product 10 may be performed in accordance with the corresponding operations of the next stage of process 100. Alternatively, in response to determining that an event did occur during the current stage of process 100 at step 204, as detected by the sensing device 18, data associated with the event may be transmitted from product 10 (and specifically via sensing device 18) to the one or more remote systems or computers (e.g., a processor) at step 208.

The transmitted data may include a plurality of information, such as, but not limited to, sensor data indicative of the parameter(s) detected by sensing device 18. In some embodiments, the sensor data may include a corresponding time period during the current stage of process 100 at which the parameter(s) was detected. In other embodiments, the sensor data may include a corresponding identification of a component of product 10 (e.g., first component 12, second component 14, third component 16) at which the parameter(s) was received. For illustrative example only, referring back to FIG. 4, the event may include a physical manipulation of product 10 (e.g., grasping) by fourth manufacturing instrument 28 while product 10 is positioned adjacent to the fourth location 38 of assembly line 30 in manufacturing environment 20. In grasping product 10, sensing device 18 may detect a load (i.e. the parameter) applied to second component 14 (see FIG. 1) by fourth manufacturing instrument 28.

Referring to FIG. 3, at step 210, an originating source or cause of the event may be determined, such as based on the sensor data detected and transmitted by sensing device 18. In the example, the originating source or cause of the event (e.g. physical manipulation of product 10) may be determined to be the fourth manufacturing instrument 28 positioned adjacent to the fourth location 38 along the assembly line 30. At step 212, a relative value or degree of the parameter applied to product 10 (e.g., the load) may be determined, such as based on the sensor data detected and transmitted by sensing device 18. In the illustrative example of FIG. 4, the value of the parameter applied to product 10 by the fourth manufacturing instrument 28 may be approximately 2 Newton (N). In other examples, the value of the parameter may be greater than or less than 2 N, or measured by various other suitable units of measurement.

It should be appreciated that multiple events may be detected during the current stage of process 100 at step 204, and/or multiple parameters may be applied to product 10 during a single event, such that the sensor data transmitted at step 208 may include data indicative of the one or more event(s) and/or parameter(s) detected during the current stage. In this instance, method 200 may include a separate determination of each source of event (at step 210) and a value or degree of each parameter (at step 212) for the one or more event(s) and/or parameter(s) detected during the current stage of process 100.

At step 214, the parameter(s) determined at step 212 may be compared to a threshold. In some embodiments, the threshold may be predetermined and stored, such as in the one or more remote systems. In other embodiments, the threshold may be user-defined and received by the one or more remote systems, such as via a user input. The threshold may define a maximum (acceptable) value or degree of the parameter determined at step 212 without necessitating modifications to process 100 for purposes of inhibiting further occurrences of the event detected at step 204. Alternatively, the threshold may define a maximum value or degree of a parameter without necessitating modifications to process 100 for purposes of minimizing further applications of the parameter to product 10 during subsequent occurrences of the event. In this instance, despite a continued occurrence of the event during further processes 100, the parameter experienced by product 10 at the event may be minimized. A value or degree of a parameter that exceeds the threshold may be indicative of a rejection parameter that is sufficient to cause a change to the product and/or at least partially impair product 10 in some form, such as a structural change to the product, a functional change to the product, a change in a level of sterility of the product, a change in a level of fluid stored in the product, and more.

For example, the threshold may define a maximum value or degree of a parameter that corresponds to conditions in which damage or defect(s) (e.g., physical, functional, aesthetic, operational, etc.) to product 10 are not occur despite the occurrence of the event at step 204. In this instance, a parameter that is determined to exceed the threshold may be indicative of a product that has experienced (or has an increased likelihood of experiencing) some form of damage or defect, while a parameter that does not exceed the threshold may be indicative of a product that has not experienced (or does not have an increased likelihood of experiencing) damage or defect.

Stated differently, a value or degree of the parameter determined at step 212 may be less than a minimum value or degree necessary to cause a resulting change or impairment (e.g., defect or damage) to product 10, with said minimum value or degree being approximately less than (or equal to) the threshold. Alternatively, the determined parameter at step 212 may be sufficient to cause a change or render product 10 at least partially damaged, defective, impaired, unsterile, and/or inoperable for its intended use, with the value or degree of the determined parameter being greater than (or equal to) the threshold.

In examples in which product 10 may include a medical device, such as a syringe or an auto-injector having one or more of a needle, a plunger, and/or a barrel storing a fluid substance therein, a parameter applied to the medical device that exceeds the threshold may cause an inadvertent movement of said components. For example, the application of a parameter exceeding the threshold may involve deflecting the needle and rendering the medical device inoperable for use by a user (e.g., a patient), moving the plunger and causing a premature release of the stored fluid substance, or breaking the barrel and causing the stored fluid substance to leak therefrom. By way of further example, the application of a parameter exceeding the threshold may involve exposing the barrel to excessive vibration, shock, light and/or heat, thereby physically and/or chemically altering the properties of the fluid substance stored therein.

In some embodiments, at least one threshold may be defined and/or predetermined for a plurality of parameters (e.g., a force, a pressure, a temperature, a light intensity, a fluid level, an impedance, etc.) that may be experienced by product 10 in process 100. Accordingly, the applicable threshold corresponding to a type of parameter (from the plurality of parameters) determined at step 212 may be identified and compared with at step 214. It should be appreciated that the threshold may include a value or degree that corresponds to a unit of measurement of the associated parameter.

Still referring to FIG. 3, whether the value or degree of the parameter exceeds the threshold may be determined at step 216. In response to determining the parameter does not exceed the threshold at step 216, method 200 may transition to step 206 in which product 10 may continue through to a next stage of process 100. In this instance, given that the parameter was not of a value or degree sufficient to cause a resulting damage or defect to product 10, method 200 may return to step 202 during which product 10 may be prepared and/or managed in accordance with the corresponding operations of the next stage of process 100. Alternatively, in response to determining the parameter does exceed the threshold at step 216, one or more proposed modifications to the current stage of process 100 may be identified at step 218, such as by the one or more remote systems.

The one or more proposed modifications identified at step 218 may be directed to resolving human or automated equipment error involved in process 100 at the current stage. For example, the one or more remote systems may be configured and operable to identify potential modifications to a position, a location, an operation, and/or various other characteristics of the originating source of the event for purposes of inhibiting further occurrences of the event (detected at step 204) and/or minimizing the value or degree of the parameter (determined at step 212) experienced by further products 10 during the current stage of process 100. Referring to the illustrative example of FIG. 4, the remote system(s) may be configured to identify modifications to one or more characteristics of the fourth manufacturing instrument 28, which was determined to be the source of the event at step 210.

In another example, the one or more remote systems may be configured and operable to identify potential modifications to objects other than the source of the event which may directly and/or indirectly influence occurrence of the event. Referring to the illustrative example of FIG. 4, the remote system(s) may be configured to identify modifications to one or more of first manufacturing instrument 22, second manufacturing instrument 24, third manufacturing instrument 26, assembly line 30, and/or one or more other surfaces, tools, assemblies, machinery, equipment, instrumentation, systems, etc.

As described in detail above, one or more objects responsible for performing operations associated with a current stage of process 100 (e.g., manufacturing, quality control testing, sterilization, packaging, storage, shipment, sale, and/or delivery) may be controlled through an automated operation (e.g., by a computer). In this instance, the potential modification(s) identified at step 218 may include adjustments to the automated control of the object, such as by modifying computer-executable instructions and/or programmable computer code operable to automate operation of the object. In embodiments in which the object may be manually operated by personnel of process 100, the potential modification(s) identified at step 218 may be communicated to the user and/or personnel for implementation.

Still referring to FIG. 3, method 200 may include step 220 in which the modifications identified for the current stage of process 100 at step 218 may be communicated to a user, such as for approval or rejection (e.g., via a user input through the one or more remote systems). In other embodiments, the one or more remote systems may be configured and operable to automatically implement the one or more modifications at step 220. Upon communicating and/or implementing the modification for the current stage of process 100 at step 220, method 200 may return to step 202 during which product 10 may be prepared and/or managed in accordance with the corresponding operations of the next stage of process 100.

It should be appreciated that the general discussion of this disclosure provides a brief, general description of a suitable computing environment in which the present disclosure may be implemented. In one embodiment, any of the disclosed systems and/or methods may be executed by or implemented by a computing system consistent with or similar to that explained in this disclosure (e.g., one or more remote systems). Although not required, aspects of the present disclosure are described in the context of computer-executable instructions, such as routines executed by a data processing device, e.g., a computer hardware platform, a wireless device, and/or a personal computer. Those skilled in the art will appreciate that aspects of the present disclosure can be practiced with other communications, data processing, or computer system configurations. Indeed, the terms “computer,” “processor,” “remote systems” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain steps of method 200, are described as being performed exclusively on a single device or system, the present disclosure may also be practiced in distributed environments where functions or modules are shared among disparate processing devices, which are linked through a communications network. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media. Alternatively, computer implemented instructions, data structures, screen displays, and other data under aspects of the present disclosure may be distributed over the Internet and/or over other networks (including wireless networks). Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, which may provide non-transitory storage at any time for the software programming. As used herein, unless restricted to non-transitory, tangible “storage” media, terms such as computer “readable medium” refer to any medium that participates in providing instructions to a processor for execution.

While a number of embodiments are presented herein, multiple variations on such embodiments, and combinations of elements from one or more embodiments, are possible and are contemplated to be within the scope of the present disclosure. Moreover, those skilled in the art will appreciate that the conception upon which this disclosure is based may readily be used as a basis for designing other devices, methods, and systems for carrying out the several purposes of the present disclosure.

Embodiments of the present disclosure may include the following features:

Item 1. A method for identifying an event during a supply chain process, comprising: preparing a product during the supply chain process; determining an occurrence of the event during preparation of the product during the supply chain process, wherein the event causes a change of the product; and identifying a modification to the supply chain process to inhibit occurrences of the event during subsequent preparations of the product in the supply chain process.

Item 2. The method of item 1, further comprising: determining a source in the supply chain process causing the occurrence of the event, wherein identifying the modification includes identifying an adjustment to an operation of the source.

Item 3. The method of item 2, wherein prior to identifying the modification, the method comprises: determining a parameter is applied to the product during the event by the source, the parameter causing damage to the product.

Item 4. The method of item 3, wherein the product includes a sensing device configured to detect the parameter applied to the product during the occurrence of the event.

Item 5. The method of item 4, wherein prior to identifying the modification, the method comprises: transmitting the parameter to a remote processor that is communicatively coupled to the sensing device, wherein the remote processor is configured to determine a value of the parameter.

Item 6. The method of item 5, wherein prior to identifying the modification, the method comprises: comparing the value of the parameter to a threshold; and identifying the modification in response to the value of the parameter exceeding the threshold, and forgoing identification of the modification in response to the value of the parameter not exceeding the threshold.

Item 7. The method of item 3, wherein the parameter includes a temperature, a pressure, a force, a velocity, a light intensity, a fluid level, an impedance, a compression, a tilt, or a rotation experienced by the product during the event.

Item 8. The method of item 4, wherein sensing device includes an accelerometer, a gyro sensor, a temperature probe, a pressure sensor, a fluid level sensor, a vaporized hydrogen peroxide (VHP) sensor, a photometer (light) sensor, a photodetector, or a chemical (biological) sensor.

Item 9. The method of item 2, wherein: the product includes a device, a tool, a machine, an instrument, a mechanism, an appliance, a gadget, or an apparatus; and the source includes a surface, a tool, an assembly, a machinery, an equipment, an instrumentation, or a system responsible for performing operations in the supply chain process to prepare the product.

Item 10. The method of item 9, wherein the product includes a medical device and the source includes an automated instrumentation for manufacturing the medical device, such that identifying the modification to the supply chain process includes an adjustment to computer-executable instructions defining an operation of the automated instrumentation.

Item 11. The method of item 10, wherein the medical device includes an autoinjector or a syringe.

Item 12. The method of item 9, wherein preparing the product during the supply chain process includes one or more of manufacturing, assembling, testing, sterilizing, packaging, labeling or marking, storing, shipping, selling, or delivering the product.

Item 13. The method of item 2, wherein the event is a first event occurring during a first stage of the supply chain process for preparing the product, and the source causing the occurrence of the event is a first source in the supply chain process for preparing the product at the first stage.

Item 14. The method of item 13, further comprising: determining an occurrence of a second event during preparation of the product during a second stage of the supply chain process, wherein the second stage is different than the first stage of the supply chain process; and determining a second source in the supply chain process causing the occurrence of the second event, wherein the second source is different than the first source.

Item 15. The method of item 14, further comprising: identifying a second modification to the supply chain process to inhibit occurrences of the second event during subsequent preparations of the product in the supply chain process.

Item 16. The method of item 1, wherein the change includes a structural change to the product, a change in a level of sterility of the product, or a change in a level of fluid stored in the product.

Item 17. A method for reducing an occurrence of an event during a supply chain process, comprising: preparing a product during the supply chain process; determining the occurrence of the event while preparing the product in the supply chain process, wherein the event causes a change to the product; and determining a modification to the supply chain process that reduces the occurrence of the event in the supply chain process during subsequent preparations of the product, thereby reducing the occurrence of the change to the product.

Item 18. The method of item 17, wherein prior to determining the modification, the method comprises: determining a source responsible for preparing the product in the supply chain process is causing the occurrence of the event; determining a parameter applied to the product by the source during the occurrence of the event; comparing a value of the parameter to a threshold; and determining the modification in response to the value of the parameter exceeding the threshold, and forgoing determination of the modification in response to the value of the parameter not exceeding the threshold.

Item 19. The method of item 18, wherein the product includes a device, a tool, a machine, an instrument, a mechanism, an appliance, a gadget, or an apparatus; wherein the source includes a surface, a tool, an assembly, a machinery, an equipment, an instrumentation, or a system responsible for performing operations in the supply chain process to prepare the product; and wherein the parameter includes a temperature, a pressure, a force, a velocity, a light intensity, a fluid level, an impedance, a compression, a tilt, or a rotation experienced by the product during the event.

Item 20. The method of item 17, wherein preparing the product during the supply chain process includes one or more of manufacturing, assembling, testing, sterilizing, packaging, labeling or marking, storing, shipping, selling, or delivering the product.

Item 21. The method of item 17, wherein the change to the product includes a structural change, a change in a level of sterility, or a change in a level of fluid stored in the product.

Item 22. A method for identifying an event causing a change to a product during a supply chain process, comprising: preparing the product during the supply chain process; determining the event occurs when preparing the product in the supply chain process, such that the product experiences the change due to the event; and identifying a modification to the supply chain process that inhibits occurrences of the change to the product during preparation by reducing the occurrence of the event in the supply chain process.

Item 23. The method of item 22, wherein prior to identifying the modification, the method comprises: determining a source of the event and a parameter applied to the product by the source that causes the change to the product; comparing the parameter to a threshold; and identifying the modification when the parameter exceeds the threshold, and forgoing identification of the modification when the parameter does not exceed the threshold.

Item 24. The method of item 22, wherein preparing the product during the supply chain process includes one or more of manufacturing, assembling, testing, sterilizing, packaging, labeling or marking, storing, shipping, selling, or delivering the product; and wherein the source includes a surface, a tool, an assembly, a machinery, an equipment, an instrumentation, or a system that performs operations in the supply chain process for preparing the product.

Item 25. The method of item 22, wherein the change to the product includes a structural change, a change in a level of sterility, or a change in a level of fluid stored in the product.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for identifying an event during a supply chain process, comprising:
preparing a product during the supply chain process;
determining an occurrence of the event during preparation of the product during the supply chain process, wherein the event causes a change of the product; and
identifying a modification to the supply chain process to inhibit occurrences of the event during subsequent preparations of the product in the supply chain process.

2. The method of claim 1, further comprising:
determining a source in the supply chain process causing the occurrence of the event, wherein identifying the modification includes identifying an adjustment to an operation of the source.

3. The method of claim 2, wherein prior to identifying the modification, the method comprises:
determining a parameter is applied to the product during the event by the source, the parameter causing damage to the product.

4. The method of claim 3, wherein the product includes a sensing device configured to detect the parameter applied to the product during the occurrence of the event.

5. The method of claim 4, wherein prior to identifying the modification, the method comprises:
transmitting the parameter to a remote processor that is communicatively coupled to the sensing device, wherein the remote processor is configured to determine a value of the parameter.

6. The method of claim 5, wherein prior to identifying the modification, the method comprises:
comparing the value of the parameter to a threshold; and
identifying the modification in response to the value of the parameter exceeding the threshold, and forgoing identification of the modification in response to the value of the parameter not exceeding the threshold.

7. The method of claim 3, wherein the parameter includes a temperature, a pressure, a force, a velocity, a light intensity, a fluid level, an impedance, a compression, a tilt, or a rotation experienced by the product during the event.

8. The method of claim 4, wherein the sensing device includes an accelerometer, a gyro sensor, a temperature probe, a pressure sensor, a fluid level sensor, a vaporized hydrogen peroxide (VHP) sensor, a photometer (light) sensor, a photodetector, or a chemical (biological) sensor.

9. The method of claim 2, wherein:
the product includes a device, a tool, a machine, an instrument, a mechanism, an appliance, a gadget, or an apparatus; and
the source includes a surface, a tool, an assembly, a machinery, an equipment, an instrumentation, or a system responsible for performing operations in the supply chain process to prepare the product.

10. The method of claim 9, wherein the product includes a medical device and the source includes an automated instrumentation for manufacturing the medical device, such that identifying the modification to the supply chain process includes an adjustment to computer-executable instructions defining an operation of the automated instrumentation.

11. The method of claim 10, wherein the medical device includes an autoinjector or a syringe.

12. The method of claim 9, wherein preparing the product during the supply chain process includes one or more of manufacturing, assembling, testing, sterilizing, packaging, labeling or marking, storing, shipping, selling, or delivering the product.

13. The method of claim 2, wherein the event is a first event occurring during a first stage of the supply chain process for preparing the product, and the source causing the occurrence of the event is a first source in the supply chain process for preparing the product at the first stage.

14. The method of claim 13, further comprising:
determining an occurrence of a second event during preparation of the product during a second stage of the supply chain process, wherein the second stage is different than the first stage of the supply chain process; and
determining a second source in the supply chain process causing the occurrence of the second event, wherein the second source is different than the first source.

15. The method of claim 14, further comprising:
identifying a second modification to the supply chain process to inhibit occurrences of the second event during subsequent preparations of the product in the supply chain process.

16. The method of claim 1, wherein the change includes a structural change to the product, a change in a level of sterility of the product, or a change in a level of fluid stored in the product.

17. A method for reducing an occurrence of an event during a supply chain process, comprising:
preparing a product during the supply chain process;
determining the occurrence of the event while preparing the product in the supply chain process, wherein the event causes a change to the product; and
determining a modification to the supply chain process that reduces the occurrence of the event in the supply chain process during subsequent preparations of the product, thereby reducing the occurrence of the change to the product.

18. The method of claim 17, wherein prior to determining the modification, the method comprises:
determining a source responsible for preparing the product in the supply chain process is causing the occurrence of the event;
determining a parameter applied to the product by the source during the occurrence of the event;
comparing a value of the parameter to a threshold; and
determining the modification in response to the value of the parameter exceeding the threshold, and forgoing determination of the modification in response to the value of the parameter not exceeding the threshold.

19. The method of claim 18, wherein the product includes a device, a tool, a machine, an instrument, a mechanism, an appliance, a gadget, or an apparatus;
wherein the source includes a surface, a tool, an assembly, a machinery, an equipment, an instrumentation, or a system responsible for performing operations in the supply chain process to prepare the product; and
wherein the parameter includes a temperature, a pressure, a force, a velocity, a light intensity, a fluid level, an impedance, a compression, a vibration, a shock, a tilt, or a rotation experienced by the product during the event.

20. The method of claim 17, wherein preparing the product during the supply chain process includes one or more of manufacturing, assembling, testing, sterilizing, packaging, labeling or marking, storing, shipping, selling, or delivering the product.

21. The method of claim 17, wherein the change to the product includes a structural change, a change in a level of sterility, or a change in a level of fluid stored in the product.

22. A method for identifying an event causing a change to a product during a supply chain process, comprising:
preparing the product during the supply chain process;
determining the event occurs when preparing the product in the supply chain process, such that the product experiences the change during the supply chain process; and
identifying a modification to the supply chain process that inhibits the change to the product by inhibiting an occurrence of the event during the supply chain process.

23. The method of claim 22, wherein prior to identifying the modification, the method comprises:
determining a source of the event and a parameter applied to the product by the source that causes the change to the product;
comparing the parameter to a threshold; and
identifying the modification when the parameter exceeds the threshold, and forgoing identification of the modification when the parameter does not exceed the threshold.

24. The method of claim 23, wherein preparing the product during the supply chain process includes one or more of manufacturing, assembling, testing, sterilizing, packaging, labeling or marking, storing, shipping, selling, or delivering the product; and
wherein the source includes a surface, a tool, an assembly, a machinery, an equipment, an instrumentation, or a system that performs operations in the supply chain process for preparing the product.

25. The method of claim 22, wherein the change to the product includes a structural change, a change in a level of sterility, or a change in a level of fluid stored in the product.
